(19) United States
(12) Patent Application Publication
(10) Pub. No.: US 2015/0099638 A1
(43) Pub. Date: Apr. 9, 2015

(54) HIGH INTERNAL PHASE COMPOSITIONS UTILIZING A GEMINI SURFACTANT

(71) Applicant: Presperse Corporation, Somerset, NJ (US)

(72) Inventors: Steve Cochran, Robbinsville, NJ (US); Masashi Yamamoto, Kanagawa (JP); Daphne Benderly, Metuchen, NJ (US); Christine Lynn Lummer, Sparta, NJ (US); Marie Yednak-Carpenter, Jackson, NJ (US)

(21) Appl. No.: 14/507,097

(22) Filed: Oct. 6, 2014

Related U.S. Application Data
(60) Provisional application No. 61/887,718, filed on Oct. 7, 2013.

(51) Int. Cl. A61K 8/06; A61K 8/85; A01N 25/04; A61K 8/68

(52) U.S. Cl. CPC A61K 8/068; A61K 8/68; A61K 8/85; A01N 25/04; A61Q 19/00
USPC 504/361; 514/772.1; 424/59; 424/401

(57) ABSTRACT

Stabilization compositions for HIP emulsions are disclosed which include a combination of a Gemini surfactant, a sugar-derived compound and a hydrophobically modified polymer. HIP emulsions are disclosed which include the stabilizing composition. Amino-acid based Gemini surfactants may be employed in the stabilizing compositions.

### CROSS-REFERENCE TO RELATED APPLICATIONS

This application claims the benefit of U.S. Provisional Patent Application No. 61/887,718 filed Oct. 7, 2013, the entirety of which incorporated herein by reference.

### FIELD OF THE INVENTION

The invention relates to high internal phase compositions, specifically, high internal phase emulsion compositions utilizing a stabilization composition including a pre-blended core of a Gemini surfactant, a sugar-derived compound and a hydrophobically modified polymer.

### BACKGROUND

High internal phase (“HIP”) emulsions have been used in a wide array of industries, including cosmetics and toiletries, agricultural products, cleaning products, and emulsified fuels.

One of the drawbacks of currently available HIP emulsions is the use of high levels of surfactants typically needed to keep the internal, or dispersed, phase adequately emulsified and stable. High levels of surfactants not only result in higher raw material costs, but can increase the likelihood of finished emulsions to cause skin irritation, especially in cosmetics, toiletries and cleaning products. For example, many HIP emulsions may use over 5% by weight of a harsh surfactant such as sodium lauryl sulfate (SLS) to sufficiently stabilize the emulsion.

Although some HIP emulsions may use sodium laureth sulfate, an ethoxylated version of SLS, to reduce the irritation potential of the surfactant, sodium laureth sulfate can lead to contamination of the resulting surfactant with 1,4-dixoane, a suspected carcinogen.

Thus there is a need for stable HIP emulsions that are less costly to produce, less irritating than currently available HIP emulsions while also being free of undesirable contaminants.

### SUMMARY OF THE INVENTION

It is an objective of the disclosed subject matter to provide a stabilization composition for HIP emulsions, the stabilization composition including a pre-blended core of a Gemini surfactant, a sugar-derived compound and a hydrophobically modified polymer.

It is a further objective to provide to industries that use HIP emulsions, such as but not limited to the cosmetics industry, a suitable HIP emulsion that uses a three part stabilization composition at a very low concentration, and contains no sulfates, ethylene oxide or propylene oxide.

It is still a further objective to provide HIP emulsions that exhibit high emulsion stability, requires low energy input to produce and is extremely versatile when used as a formulating tool.

In accordance with a first embodiment, a HIP emulsion stabilization composition is disclosed which includes a blended composition of a Gemini surfactant, a sugar-derived compound and a hydrophobically modified polymer. The stabilization composition, when used as part of a HIP emulsion, unexpectedly and significantly increases the stability and versatility of HIP emulsions and HIP gels more effectively than previously disclosed compositions.

Without being confined to a single theory, this increased stability is thought to be due to the synergistic interactions of the Gemini surfactant, sugar-derived compound and polymer, which may be intensified by intimate mixture before combination with the remaining components of the HIP emulsion. It has been found that only extremely, unexpectedly low levels of the stabilization compositions are needed to provide highly stabilized HIP emulsions and gels, providing several advantages over other surfactant systems. Accordingly, among the advantages achieved are a near-zero surfactant level which greatly minimizes the potential for the finished HIP emulsion containing the stabilization composition to cause irritation. Moreover, the compositions are sulfate-free, ethylene oxide-free and propylene oxide-free, have very low process-energy requirements and low temperature requirements for manufacturing. HIP emulsions employing the disclosed stabilization compositions are less costly to produce and less irritating than currently available HIP emulsion systems while also being free of undesirable contaminants. The low energy and temperature process requirements offer a more responsible energy conservation manufacturing alternative to current manufacturing methods. Other advantages include renewable chemistries based on natural amino acids and sugar, the potential to produce transparent gel compositions if so desired, easy substitution of the internal phase (i.e., dispersed) components of the emulsion and the possibility of extensive dilution of the emulsion with water.

In accordance with some embodiments the stabilizing composition includes 1 to 15 wt. % Gemini surfactant, 70 to 98 wt. % sugar-derived compound and 1 to 15 wt. % hydrophobically modified polymer. In other embodiments, the stabilizing composition consists essentially of 1 to 15 wt. % Gemini surfactant, 70 to 98 wt. % sugar-derived compound and 1 to 15 wt. % hydrophobically modified polymer. In other embodiments the stabilizing composition may consist only of 1 to 15 wt. % Gemini surfactant, 70 to 98 wt. % sugar-derived compound and 1 to 15 wt. % hydrophobically modified polymer. The stabilizing composition may be sulfate-, ethoxylate- and propoxylate-free, and/or the Gemini surfactant may be at least one amino-acid based Gemini surfactant such as sodium dilauramidoglutamide lysine.

The sugar-derived compound may include one or more of sodium hydroxypropylsulfonate laurylglucosides crosspolymer, polyglycerols and glucose esters.

The hydrophobically modified polymer may include one or more of hydrophobically modified acrylates, polyacrylates and polyethers.

In accordance with another embodiment, disclosed are sulfate-, ethoxylate- and propoxylate-free HIP emulsions including a low concentration of a stabilization composition which includes an amino acid-based Gemini surfactant, a sugar-derived compound and a hydrophobically modified polymer. The subject HIP emulsions are easy to manufacture, versatile, highly stable and are not harmful to the environment.

In a further embodiment, disclosed are HIP emulsions containing a hydrocarbon liquid as the internal phase, water and glycerin as the continuous phase, and a stabilizing composition including an amino acid-based Gemini surfactant, a sugar-derived compound and a hydrophobically modified polymer.

In other embodiments, HIP emulsion compositions may include 0.2 to 5 wt. % of a stabilizing composition as disclosed herein, 15 to 35 wt. % of a continuous phase, and 65 to 80 wt. % of an internal phase. The continuous phase may be water, glycerin or a combination of water and glycerin. The composition may include 5-20 wt. % glycerin.

The internal phase may include hydrocarbons, silicones or silicon-based materials, triglycerides, waxes, esters and/or fatty acids.

In some embodiments HIP emulsions disclosed herein have an average droplet size from about 2 to about 30 μm. In other embodiments the compositions have an average droplet size is less than 20 μm.

In one embodiment, the HIP emulsion may have the appearance of a clear gel when the refractive indices of the two phases have been matched by adjusting the ratio of the ingredients. However, other embodiments may not appear as a clear gel.

In other embodiments products including the disclosed compositions include cosmetics, body care products, hair care products, bath products, sunscreen products, and agricultural products.

In further embodiments, methods of making a HIP emulsion as disclosed herein include combining a Gemini surfactant, a sugar-derived compound and a hydrophobically modified polymer to form a stabilizing component, combining the stabilizing component with a continuous phase, and combining a resulting combination with an internal phase.

### BRIEF DESCRIPTION OF THE DRAWINGS

So that those having ordinary skill in the art will have a better understanding of how to make and use the disclosed systems and methods, reference is made to the accompanying figures wherein:

FIG. 1 is a graphical depiction of a comparison of droplet size in accordance with one or more embodiments of the presently disclosed subject matter.

### DETAILED DESCRIPTION OF THE INVENTION

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

### Stabilizing Compositions

Stabilizing compositions for use in HIP emulsions are disclosed including a Gemini surfactant, a sugar-derived compound and a hydrophobically modified polymer. Stabilizing compositions in accordance with the present disclosure may contain no sulfates, ethylene oxide and/or propylene oxide.

As used herein “high internal phase” (HIP), with respect to oil-in-water emulsions, means and includes emulsions in which the internal phase makes up at least 65 wt. % of the volume of the emulsion.

Gemini surfactants, sometimes called dimeric surfactants, have two hydrophilic head groups and two hydrophobic groups in the molecules, in contrast to conventional surfactants that generally have a single hydrophilic head group and a single hydrophobic group in the molecule. Gemini surfactants can be ten to a thousand times more surface active than conventional surfactants with similar but single hydrophilic and hydrophobic groups in the molecules. Gemini surfactants are considerably more surface-active than conventional surfactants, and have remarkably low CMC values compared to the corresponding conventional surfactants of equivalent chain length. Examples of suitable Gemini surfactants that may be employed in the subject stabilization systems include sodium dilauramidoglutamide lysine, sodium cocoyl ethylene diamine PEG-15, etc. One or more Gemini surfactants may be combined to form the Gemini surfactant component of the disclosed stabilizing compositions.

In one embodiment, amino acid-based Gemini surfactants may be employed. As used herein “amino-acid-based Gemini surfactant” means and includes Gemini surfactants which are derived from an amino acid and/or contain one or more amino acids. Examples of suitable amino acid-based Gemini surfactants include sodium dilauramidoglutamide lysine, sodium cocoyl ethylene diamine PEG-15, etc. In one or more embodiments, the amino acid-based surfactant is Pellicer™ L30 Gemini surfactant commercially available from Asahi Kasei Corp. of Tokyo, Japan. The amount of Gemini surfactant in the stabilizing composition by weight percent is from 1 to 15 wt. %. In some embodiments the amount of Gemini surfactant present is from 3 to 8 wt. %. In other embodiments the amount of Gemini surfactant present is from 4 to 6 wt. %.

Sugar-derived compounds which may be employed include for example sodium hydroxypropylsulfonate laurylglucosides crosspolymer, polyglycerols, (such as polyglyceol-3, polyglycerol-6 distearate, polyglycerol-4 laurate, etc.), glucose esters (such as methyl glucose sesquistearate), etc. As used herein “sugar-derived compounds” means and includes compounds which have one or more 6 membered ring structures, or mono-, di- or tri-carboxyester substituted methyl groups. One or more sugar-derived compounds may be combined to form the sugar-derived compound component of the stabilizing composition. In one or more embodiments, the sugar-derived compound is PolySugaNate 160P sodium hydroxypropylsulfonate laurylglucosides crosspolymer commercially available from Colonial Chemical of South Pittsburgh, Tenn. The amount of sugar-derived compound in the stabilizing composition by weight percent is from 70 to 98 wt. %. In some embodiments the amount of sugar-derived compound present is from 80 to 95 wt. %. In other embodiments the amount of sugar-derived compound present is from 87 to 92 wt. %.

Hydrophobically modified polymers which may be employed in the subject stabilization compositions include for example acrylates, polyacrylates, polyethers, etc. As used herein “hydrophobically modified polymers” means and includes compounds in which a hydrophobe is attached to the hydrophilic polymer. This creates a hydrophilic and hydrophobic part of the new molecule, which can now act as a surfactant, or bridge between the oil and water phases. It can include hydrophobically modified acrylates, cationic acrylates and hydrophobically modified polyethers. One or more hydrophobically modified polymers may be combined to form the Gemini hydrophobically modified polymer component of the stabilizing composition. In one or more embodiments, the hydrophobically modified polymer is a C10/30 alkyl acrylate crosspolymer such as Aqupec SER W300C available commercially from Sumitomo Seika, Osaka, Japan. The amount of hydrophobically modified polymer in the stabilizing composition by weight percent is from 1 to 15 wt. %. In some embodiments the amount of hydrophobically modified polymer present is from 3 to 8 wt. %. In other embodiments the amount of hydrophobically modified polymer present is from 4 to 6 wt. %.

In some embodiments, stabilizing compositions as disclosed herein may consist only of a Gemini surfactant, a sugar-derived compound and a hydrophobically modified polymer. In such embodiments, the compositions may include from 1 to 15 wt. % of Gemini surfactant, from 1 to 15 wt. % of hydrophobically modified polymer, and from 70 to 98 wt. % sugar derived compound. In other embodiments, stabilizing compositions may include one or more additives or ingredients that do not or would not materially affect the basic and novel properties of the stabilizing composition, and/or the performance of the stabilizing composition as a stabilizer in a HIP. For example, certain additives, such as fragrances, preservatives, colorants, antioxidants, film-formers, humectants, etc. will not affect the performance of stabilizing compositions disclosed herein. Thus, stabilizing compositions are disclosed which consist essentially of a Gemini surfactant, a sugar-derived compound and a hydrophobically modified polymer system.

The inventors have surprisingly found that use of from 0.03 wt. % to 0.05 wt. % of Gemini surfactants disclosed herein is adequate to form stable HIP emulsions and gels. This stability is easily shown by visible phase separation differences which typically show up within days at either room temperature or elevated temperatures. By comparison, HIP emulsions typically require from at least 0.5 to 5.0 wt. % of any commercially available surfactant to achieve adequate stability, representing a difference of at least an order of magnitude.

### HIP Emulsions Including a Stabilizing Composition

HIP emulsions may be prepared using the above-disclosed stabilizing compositions. HIP emulsions in accordance with the present disclosure may include from about 0.2 to about 5 wt. % of the stabilizing composition.

The remaining components of the HIP emulsion include an external, or continuous, phase, and an internal, or dispersed, phase. Optional further components may include one or more preservatives, colorants, fragrances, oils, active ingredients, etc.

The continuous phase may include one or more of water, glycerin, or other polyols such as sorbitol, etc. HIP emulsions as described herein include from 20 to 35 wt. % continuous phase. In some embodiments the HIP emulsion may include from 21 to 28 wt. % continuous phase. The internal phase may include one or more oils, waxes, silicones, triglycerides, esters, fatty acids or hydrocarbons, providing innumerable variations that may be used in the disclosed HIP emulsions. HIP emulsions as described herein include from 65 to 80 wt. % internal phase. In some embodiments the HIP emulsion may include from 72 to 79 wt. % internal phase.

Non-limiting examples of oils that may be employed in the internal phase include coconut oil, jojoba oil, castor oil, almond oil, etc.

Non-limiting examples of waxes that may be employed in the internal phase include beeswax, microcrystalline wax, paraffin wax, etc.

Non-limiting examples of silicones and/or silicon-based materials that may be employed in the internal phase include cyclomethicone, cyclopentasiloxane, dimethicone, polysilanes, etc.

Non-limiting examples of triglycerides that may be employed in the internal phase include capric triglycerides, caprylic triglycerides, C10-C18 triglycerides, etc.

Non-limiting examples of esters that may be employed in the internal phase include isopropyl myristate, isopropyl palmitate, C13-C15 alkyl benzoate, etc.

Non-limiting examples of fatty acids that may be employed in the internal phase include coconut fatty acids, lanolin fatty acids, etc. Non-limiting examples of hydrocarbons that may be employed as the internal phase include isoeicosane, isododecane, polyisobutene, etc. The use of hydrocarbons as the internal phase is a common practice in cosmetics, and is appropriate for use in HIP emulsions as disclosed herein. Various known hydrocarbons may be employed as the internal phase. Hydrocarbons encompass a wide variety of chemical structures and therefore, a variety of different properties. Some commercial hydrocarbons are derived directly from fractionated petroleum, while others are built synthetically from very pure building blocks of a carefully produced specific fraction such as polyisobutenes. Within these groupings the most widely used family of hydrocarbons is the alkanes. Alkanes are fully saturated hydrocarbons that can exist in straight-chained structures (n-alkanes), branched structures (isoalkanes) and cyclic structures (cycloalkanes.) The alkanes show excellent stability due to the absence of carbon-carbon double bonds. Within these groups, the isoalkanes are widely used by cosmetic formulators and are exemplified by the line of Permethyl® hydrocarbons from Presperse Corp. (manufactured by INEOS) and the Isopars® isoalkanes from ExxonMobile. These materials come in a range of carbon chain-lengths, with the smallest chains showing volatility, with increasingly less volatility as they increase in length. Whereas isododecane (C12) is widely employed for its volatility, isohexadecane (C16) and isoeicosane (C20) are significantly less volatile, and are more widely employed for their nonvolatile properties. These hydrocarbons are widely used for their emollient properties in creams and lotions, long-wearing properties in lipsticks and waterproofing properties in sunscreens.

Another branch of hydrocarbons are represented by “mixed” alkanes and exemplified by the line of Gemseal® products from Total Petrochemicals, ranging from C13-15 alkanes to C18-21 alkanes. These materials, unlike the isoparaffins, contain various amounts of cycloalkanes and n-alkanes as well as the branched chained isoparaffins. Because of their mixed chemistries, they demonstrate different aesthetic properties than the pure isoalkanes. They are typically promoted as “non-volatile” hydrocarbons that produce emolliency, water-proofing and long-wear effects.

The skilled artisan will recognize additional suitable materials known in the art may be used and can be found listed in the INCI Dictionary (International Nomenclature of Cosmetic Ingredients) published by the Personal Care Products Council, Washington, D.C.

Non-limiting examples of optional preservatives which may be added to the HIP emulsion are methyl paraben, propyl paraben, DMDM hydantoin, and sodium benzoate. Additional examples of suitable preservatives can be found listed in the INCI Dictionary.

The following non-limiting examples and formulations serve to further illustrate embodiments of the disclosed subject matter.

### Examples

Formula 1: In accordance with one exemplary embodiment, a HIP emulsion was prepared using the components in Table 1.

TABLE 1
Formula 1

| Component | Material | Wt. % |
|---|---|---|
| Stabilizing | | |
| Composition | | |
| Gemini surfactant | sodium dilauramidoglutamide lysine | 0.05% |
| | (Pellicer L30) | |
| Sugar-derived | sodium hydroxypropylsulfonate | 0.95% |
| compound | laurylglucosides crosspolymer | |
| | (SugaNate 160P) | |
| Hydrophobically | C10/30 alkyl acrylate crosspolymer | 0.06% |
| modified polymer | (Aqupec SER W300C) | |
| Other HIP | | |
| Emulsion | | |
| Components | | |
| Preservative | phenoxyethanol and ethylhexylglycerin | 0.03% |
| | (Euxyl PE 9010, Shulke Inc.) | |
| Water | Distilled water | 2.91% |
| (continuous phase) | | |
| Glycerin | USP glycerin | 20% |
| (continuous | | |
| phase) | | |
| Internal (dispersed) | isohexadecane (Permethyl 101A, | 76% |
| phase | Presperse Corp.). | |
| | | 100.00 |

Formula 1 was prepared as follows. The Gemini surfactant, sugar-derived compound and hydrophobically modified polymer system are blended to form the stabilizing composition. The stabilizing composition is then added to the glycerin and water. The isohexadecane is added to the batch while mixing, and mixing continues until uniformity is reached. A highly stable gel is formed.

Sodium dilauramidoglutamide lysine amino acid-based Gemini surfactant, C10/30 alkyl acrylate crosspolymer hydrophobically modified polymer and sodium hydroxypropylsulfonate laurylglucosides crosspolymer sugar-derived compound together form the stablilizing composition of Formula 1, and provide structure and stability to the system. The stabilizing composition, present at only 1.06%, is adequate to provide a desirable stability to the HIP emulsion. Notably, only 0.05 wt. % Gemini surfactant is employed, which is far less than what is typically needed to provide a stable emulsion, e.g., at least 0.5 wt. %.

Water and glycerin are used in combination as the continuous phase, allowing modification of the ratio of these two ingredients to increase or decrease the refractive index of the compound. This can be done to match the refractive index of the changeable internal phase, to produce a transparent emulsion. Isohexadecane is used as the internal phase.

In Formula 1, the phenoxyethanol and ethylhexylglycerin are used in combination as an optional preservative system against microbial growth.

Formulas 2 and 3—Substitution of the Oil Phase Component

With reference to Table 2, exemplary Formulas 2 and 3 are disclosed. Formulas 2 and 3 are essentially the same, except for the substitution of isohexadecane for isoeicosane in Formula 3. The substitution of the oil phase component does not modify the physical stability of the formula.

TABLE 2
Formulas 2 and 3

| Ingredients | Formula 2 | Formula 3 |
|---|---|---|
| Isoeicosane (Permethyl 102A) | 73.97 | — |
| Isohexadecane (Permethyl 101A) | — | 73.97 |
| USP Glycerin | 12 | 12 |
| Water | 12.98 | 12.98 |
| Acrylates/C10-30 Alkyl Acrylate Crosspolymer | 0.05 | 0.05 |
| Sodium Hydroxypropylsulfonate Laurylglucoside | 0.95 | 0.95 |
| Crosspolymer (Polysuganate 160P) | | |
| Sodium Dilauramidoglutamide Lysine (Pellicer | 0.05 | 0.05 |
| L-30) | | |
| | 100.00 | 100.00 |

Formula 4—Dilution of Formula 3 with water

With reference to Table 3, Formula 4 is a formulation in which the addition of 50% additional water to Formula 3 provides a thinner, sprayable formula that retains the base formula's emulsion stability.

TABLE 3
Formula 4

| | Weight |
|---|---|
| Ingredients | % |
| Isohexadecane | 49.31 |
| USP Glycerin | 8.0 |
| Water | 42.0 |
| Acrylates/C10-30 Alkyl Acrylate Crosspolymer | 0.03 |
| Sodium Hydroxypropylsulfonate Laurylglucoside | 0.61 |
| Crosspolymer (Polysuganate 160P) | |
| Sodium Dilauramidoglutamide Lysine (Pellicer | 0.03 |
| L-30) | |
| | 100.00 |

Formulas 5 and 6—Skin Care Lotions

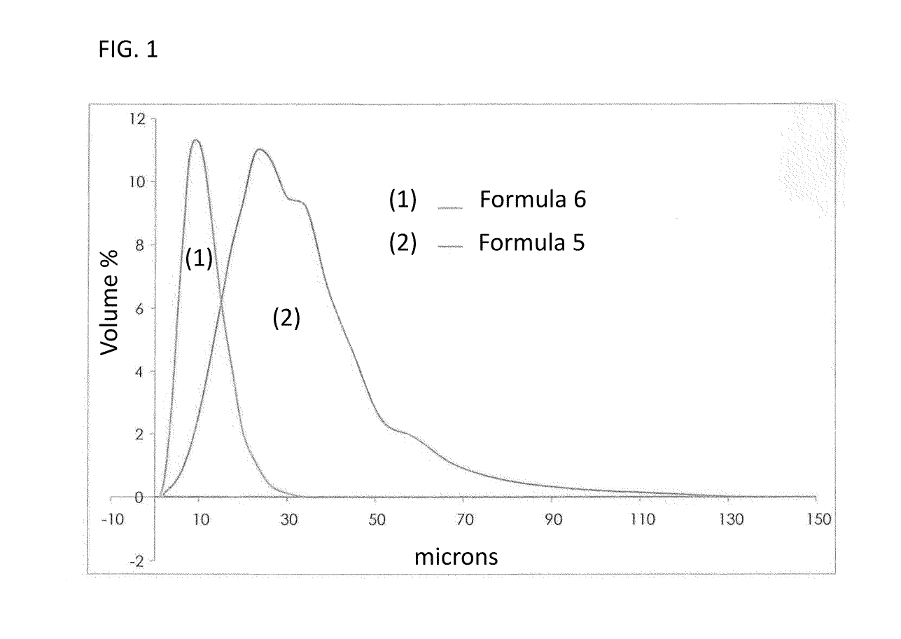

With reference to Table 4, Formula 5 was prepared in a traditional manner by combining ingredients into a water phase and an oil phase separately, then adding the oil phase to the water phase with mixing. Formula 6, which employs the pre-made HIP Gel technology was prepared by first intimately pre-blending the HIP Gel core ingredients of Polysuganate 160P, Pellicer L-30 and acrylate crosspolymer to form the stabilizing composition, adding that intimately blended stabilizing composition to the water and glycerin phase, then adding the isoeicosane with mixing. The pre-made HIP Gel composition is shown in Table 5. The other ingredients in the formula were then added to the completed HIP Gel with mixing. Now referring to FIG. 1, the HIP Gel Emulsion (Formula 6) had a much smaller and more consistent emulsion droplet size, leading to significantly more emulsion stability. Formula 6 proved stable at 50° C. for 8 weeks, while the emulsion prepared in the traditional manner (Formula 5) proved unstable at 50° C. after only 1 week.

TABLE 4
Formulas 5 and 6

| | Formula 5 | |
|---|---|---|
| | (Prepared | Formula 6 |
| | with | (Prepared using |
| | Traditional | HIP Gel |
| | Methods | Technology) |
| Ingredient Name | Weight % | Weight % |
| Water | 10.10 | 10.10 |
| Disodium EDTA | 0.01 | 0.01 |
| Lactic Acid | 0.10 | 0.10 |
| USP Sodium Chloride | 0.90 | 0.90 |
| Premade HIP Gel (*) | 0 | 78.58 (*) |
| Isoeicosane (Permethyl 102A) | 58.07 | — |
| USP Glycerin | 17.27 | — |
| Sodium Hydroxide (20%) | 0.10 | — |
| Water | 2.30 | — |
| Acrylates/C10-30 Alkyl Acrylate | 0.05 | — |
| Crosspolymer | | |
| Sodium Hydroxypropylsulfonate | 0.75 | — |
| Laurylglucoside Crosspolymer | | |
| (Polysuganate 160P) | | |
| Sodium Dilauramidoglutamide Lysine | 0.04 | — |
| (Pellicer L-30) | | |
| Sodium Hydroxide (20%) | 0.02 | 0.02 |
| Coconut Oil | 1.50 | 1.50 |
| Polyisobutene (Permethyl 104A) | 0.90 | 0.90 |
| Fragrance | 0.10 | 0.10 |
| Water and Acrylates/C10-30 Alkyl | 7.44 | 7.44 |
| Acrylate Crosspolymer (Acqua Pelle AP- | | |
| 300) | | |
| Sodium Hydroxide (20%) | 0.2 | 0.2 |
| Phenoxyethanol, Ethylhexylglycerin | 0.15 | 0.15 |
| (Euxyl PE9010) | | |
| | 100.00 | 100.00 |

TABLE 5
(*) The Premade HIP Gel Components

| Premade HIP Gel ingredients | Weight % |
|---|---|
| Isoeicosane (Permethyl 102A) | 73.90 |
| USP Glycerin | 21.98 |
| Sodium Hydroxide (20%) | 0.13 |
| Water | 2.93 |
| Acrylates/C10-30 Alkyl Acrylate | 0.06 |
| Crosspolymer | |
| Sodium Hydroxypropylsulfonate | 0.95 |
| Laurylglucoside Crosspolymer | |
| (Polysuganate 160P) | |
| Sodium Dilauramidoglutamide Lysine | 0.05 |
| (Pellicer L-30) | |
| | 100.00 |

Droplet sizes of the internal phase of the emulsion of Formula 5 were measured against those of Formula 6. A Mastersizer 2000 (Malvern Instruments) particle analyzer was used to evaluate droplet sizes. With reference to FIG. 1, in the corresponding particle size curves, use of the stabilizing system as disclosed herein leads to a much smaller and more uniform droplet size, even when diluted, which accounts for the surprising increase in stability, even at elevated temperatures.

HIP emulsions such as those of Formulas 1-4 and 6-8 which include the disclosed stabilizing composition show unexpected stability. Such compositions may be made transparent by adjusting the ratios of the ingredients so that the refractive indices of the two phases match. The compositions are ready to uses as a clear or opaque gel, or can be diluted with water to create semi-solid compositions such as creams, lotions, etc., or diluted further to create very low viscosity sprayable compositions. HIP emulsions disclosed herein are highly structured, even in the diluted form, allowing the compositions to suspend anything from air bubbles to sand, glitter, titanium dioxide, solid active ingredients, insoluble liquids or pearlescent pigments. This may permit the emulsion to function well as a decorative, transparent composition, or as an opaque composition that can function as a facial scrub or sunscreen base for inorganic actives such as titanium dioxide and zinc oxide.

Another unexpected and surprising result provided by the subject compositions is the ability of the transparent emulsion, which may be a transparent gel, to contain relatively high levels of fragrance oils, without compromising transparency, stability or structure of the gel. As a result, compositions disclosed herein are particularly effective as a unique fragrance delivery system in personal care, home fragrance products, or industrial deodorizers.

The compositions disclosed herein may be used “as is” or can be used by a formulator as an “Integrated Formulation,” which becomes the primary building block of a series of related, and easily modified product forms. Applications for the subject compositions include many categories such as cosmetics, body care products, hair care products, bath products, sunscreen products, agricultural products and the like. For example, cosmetic product forms which may employ the subject compositions include fragrances, makeup, etc. Exemplary body and skin care products include scrubs, tanning lotions and sprays, moisturizers, wipes etc. Bath products may include bath oils, body washes, shower gels, etc. Bath oil products employing the HIP emulsions using the disclosed stabilizing systems will easily disperse in bathwater for both human and animal use. Hair products may include shampoos, conditioners, styling sprays, mousses and gels, moisturizers, etc. The compositions can provide shine improvement and frizz control in the hair care area due to the high internal phase and low level of irritants. The compositions are well-suited to sunscreen products as well, since the high internal phase, when spread on the skin, will invert and form smooth oil films, conferring water-repellency or water-proofing which is important in sunscreen products.

Because the stabilizing system provides such a robust stabilized composition even when used at extremely low concentrations, the internal phase of HIP emulsions employing the stabilizing system lends itself to easy dilution. Accordingly, the stabilizing system is particularly useful in applications in which active ingredients are employed as the internal phase. For example, pesticide and fertilizer active ingredients may be employed as the internal phase of HIP emulsions including the disclosed stabilizing system. Such compositions can be diluted in the field with water, and still provide stable emulsions for agricultural application.

Moreover, the skilled artisan will recognize that active ingredients which may form suspensions in a continuous phase can be used as the internal phase in HIP emulsion compositions including the stabilizing compositions disclosed herein. For example, sunscreen active ingredients such as avobenzone, octinoxate, octocrylene, etc. can be employed as the internal phase in such HIP emulsions, as shown in Formula 7.

TABLE 6
Formula 7-Sunscreen-based HIP Gel with Sunscreen ingredient
Octinoxate

| | Weight |
|---|---|
| Ingredients | % |
| Isohexadecane | 51.47 |
| Octinoxate | 22.5 |
| USP Glycerin | 12 |
| Water | 12.98 |
| Acrylates/C10-30 Alkyl Acrylate Crosspolymer | 0.05 |
| Sodium Hydroxypropylsulfonate Laurylglucoside | 0.95 |
| Crosspolymer (Polysuganate 160P) | |
| Sodium Dilauramidoglutamide Lysine (Pellicer | 0.05 |
| L-30) | |
| | 100.00 |

Similarly, natural oils such as jojoba oil, almond oil, sunflower seed oil, etc. may be employed in such HIP emulsions, as shown in Formula 8.

TABLE 7
Formula 8 - Bath Oil-based HIP Gel using Jojoba Oil

| | Weight |
|---|---|
| Ingredients | % |
| Jojoba Oil | 73.97 |
| USP Glycerin | 12 |
| Water | 12.98 |
| Acrylates/C10-30 Alkyl Acrylate Crosspolymer | 0.05 |
| Sodium Hydroxypropylsulfonate Laurylglucoside | 0.95 |
| Crosspolymer (Polysuganate 160P) | |
| Sodium Dilauramidoglutamide Lysine (Pellicer L- | 0.05 |
| 30) | |
| | 100.00 |

In accordance with further embodiments, HIP emulsions disclosed herein may be used in sulfate-free, ethylene oxide-free and/or propylene oxide-free shampoo, conditioner and hair primer formulations. In addition to being free of the aforementioned compounds which can cause irritation, the extremely low surfactant levels present in such formulation provide the benefits of fading protection, frizz control, curl definition, and reduced drying time.

Although the compositions and systems of the present disclosure have been described with reference to exemplary embodiments thereof, the present disclosure is not limited thereby. Indeed, the exemplary embodiments are implementations of the disclosed compositions and systems are provided for illustrative and non-limitative purposes. Changes, modifications, enhancements and/or refinements to the disclosed compositions and systems may be made without departing from the spirit or scope of the present disclosure. Accordingly, such changes, modifications, enhancements and/or refinements are encompassed within the scope of the present invention.

What is claimed is:

1. A stabilizing composition for a high internal phase emulsion, the stabilizing composition comprising a Gemini surfactant, a sugar-derived compound and a hydrophobically modified polymer.

2. The composition of claim 1 comprising 1 to 15 wt. % Gemini surfactant, 70 to 98 wt. % sugar-derived compound and 1 to 15 wt. % hydrophobically modified polymer.

3. The composition of claim 1 consisting essentially of 1 to 15 wt. % Gemini surfactant, 70 to 98 wt. % sugar-derived compound and 1 to 15 wt. % hydrophobically modified polymer.

4. The composition of claim 1 consisting of 1 to 5 wt. % Gemini surfactant, 70 to 98 wt. % sugar-derived compound and 1 to 5 wt. % hydrophobically modified polymer.

5. The composition of claim 1 which is sulfate-, ethoxylate- and propoxylate-free.

6. The composition of claim 1 wherein the Gemini surfactant comprises at least one amino-acid based Gemini surfactant.

7. The composition of claim 6 wherein the at least one Gemini surfactant is sodium dilauramidoglutamide lysine.

8. The composition of claim 1 wherein the sugar-derived compound is selected from the group consisting of sodium hydroxypropylsulfonate laurylglucosides crosspolymer, polyglycerols, glucose esters and mixtures thereof.

9. The composition of claim 1 wherein the hydrophobically modified polymer is selected from the group consisting of hydrophobically modified acrylates, polyacrylates, polyethers, and mixtures thereof.

10. A high internal phase emulsion composition comprising 0.2 to 5 wt. % of the stabilizing composition of claim 1, 15 to 35 wt. % continuous phase, and 65 to 80 wt. % internal phase.

11. The composition of to claim 10 wherein the continuous phase is selected from the group consisting of water, glycerin and a combination of water and glycerin.

12. The composition of claim 11 comprising 5-20 wt. % glycerin.

13. The composition of claim 10 wherein the internal phase comprises at least one hydrocarbon.

14. The composition of claim 10, wherein the internal phase is selected from the group consisting of isoeicosane, isododecane, and polyisobutene

15. The composition of claim 10 wherein the internal phase comprises a silicon-based material.

16. The composition of claim 10, wherein the internal phase comprises at least one triglyceride.

17. The composition of claim 10, wherein the internal phase comprises at least one wax.

18. The composition of claim 10, wherein the internal phase comprises at least one ester.

19. The composition of claim 10, wherein the internal phase comprises at least one fatty acid.

20. A cosmetic composition comprising the composition of claim 10.

21. A body care product comprising the composition of claim 10.

22. A hair care products comprising the composition of claim 10.

23. A bath product comprising the composition of claim 10.

24. A sunscreen product comprising the composition of claim 10.

25. An agricultural product comprising the composition of claim 10.

26. A composition according to claim 10 having an average droplet size from about 2 to about 30 μm.

27. A composition according to claim 10 having an average droplet size less than 20 μm.

28. A method of making a HIP emulsion comprising combining a Gemini surfactant, a sugar-derived compound and a hydrophobically modified polymer to form a stabilizing component, combining the stabilizing component with a continuous phase, and combining a resulting combination with an internal phase.
